(19) United States
(12) Patent Application Publication
(10) Pub. No.: US 2022/0236294 A1
(43) Pub. Date: Jul. 28, 2022

(54) Methods for Evaluation and Treatment of Alzheimer's Disease and Applications Thereof

(71) Applicant: Huntington Medical Research Institutes, Pasadena, CA (US)

(72) Inventors: Alfred N. Fonteh, Quartz Hill, CA (US); Michael G. Harrington, Pasadena, CA (US); Katherine Jane Hamblin, Pasadena, CA (US)

(73) Assignee: Huntington Medical Research Institutes, Pasadena, CA (US)

(21) Appl. No.: 17/596,572

(22) PCT Filed: Jun. 11, 2020
(86) PCT No.: PCT/US2020/037310

§ 371 Date: Dec. 13, 2021

Related U.S. Application Data
(60) Provisional application No. 62/860,672, filed on Jun. 12, 2019.

(51) Int. Cl. G01N 33/92

(52) U.S. Cl. CPC G01N 33/92; G01N 2560/00; G01N 2800/50; G01N 2800/2821

(57) ABSTRACT

Methods to determine risk of Alzheimer's disease and applications thereof are described. Generally, systems and methods utilize analyte measurements, such as dicarboxylic acid levels, to determine a risk of Alzheimer's disease. Based on Alzheimer disease risk, diagnostics or treatments can be performed.

### CROSS REFERENCE TO RELATED APPLICATIONS

This application claims priority to U.S. Provisional Application Ser. No. 62/860,672, entitled “Methods for Evaluation and Treatment of Alzheimer's Disease and Applications Thereof” to Fonteh et al., filed Jun. 12, 2019, which is incorporated herein by reference in its entirety.

### TECHNICAL FIELD

The disclosure is generally directed to processes that evaluate risk of developing Alzheimer's Disease and applications thereof, and more specifically to methods and systems for evaluating lipid metabolites associated with Alzheimer's Disease and applications thereof, including treatments.

### BACKGROUND

Alzheimer's disease (AD) is the most common form of dementia, the sixth leading cause of death in the US, and the fourth leading cause of death in African Americans. AD is characterized by extracellular β-amyloid deposition in the brain, followed by intracellular neurofibrillary tangles of hyperphosphorylated tau proteins, accompanied by neuronal loss. All attempts to reduce amyloid deposition in dementia have been unsuccessful in preventing or slowing neurodegeneration and cognitive function, thus efforts are now focused on treatment at earlier stages of pathology. However, methods to select patients with early AD pathology are limited by incomplete understanding of early pathophysiology and lack of biomarkers to predict the onset of AD in a cognitively healthy (CH) individual. Aims to improve this selection process include clinical trials in mutation carriers with autosomal dominant AD, whose estimated clinical onset is more reliable based on each person's family history. This early onset disorder is rare and pathologically distinct from sporadic AD, for which the lack of non-invasive, widely usable, predictive biomarkers is a substantial bottleneck for properly designing trials in individuals prior to symptom onset.

The principal validated biomarkers for AD rely heavily on molecular changes in the known amyloid/tau pathology of AD, represented by decreased β-amyloid and increased tau in cerebrospinal fluid (CSF), and/or increased brain amyloid or tau by positron emission tomography (PET). These techniques are not widely available or applicable to many patients due to the invasiveness of CSF collection and PET imaging, the high expenses for these procedures and, although useful to distinguish clinical groups, they might have 10-20 years inaccuracy for predicting onset of clinical deterioration. Other candidate biomarkers from invasive studies include CSF proteins, blood measures of tau or amyloid, metabolites, or exosomes; and from non-invasive urine collection, neural thread protein. None of these preliminary candidates have been accepted or validated, and the need for more predictive molecular biomarkers is still widely recognized.

### SUMMARY OF THE INVENTION

Many embodiments are directed to methods of determining an individual's risk for Alzheimer's disease based on their dicarboxylic acid amounts. In many of these embodiments, a biological sample is obtained from the individual and the dicarboxylic acid amount in the biological sample is determined. Various embodiments are also directed towards further diagnostic testing and treatments based for individuals with high risk of Alzheimer's disease.

In an embodiment, a method is to determine an individual's risk of Alzheimer's disease. The method obtains a biological sample of an individual, wherein the biological sample contains dicarboxylic acids. The method adds an internal standard of dicarboxylic acid molecules to the biological sample. And the method performs an assay on the biological sample to determine an amount of at least one long dicarboxylic acid species in the sample. The determined amount of the at least one long dicarboxylic acid species indicates the individual's risk of Alzheimer's disease.

In another embodiment, the biological sample is urine.

In yet another embodiment, the assay is gas chromatography combined with mass spectrometry.

In a further embodiment, the method further converts the dicarboxylic acids within the biological to dipentafluorobenzyl esters prior to performing gas chromatography combined with mass spectrometry.

In still yet another embodiment, the internal standard of dicarboxylic acid molecules includes succinic acid (C4), glutaric acid (C5), pimelic acid (C7), suberic (C8), azelaic acid (C9) or sebacic acid (C10).

In yet a further embodiment, the internal standard of dicarboxylic acid molecules is a set of deuterated dicarboxylic acid molecules with known concentrations.

In an even further embodiment, the amount of at least one long dicarboxylic acid species is a relative amount to a set of one or more dicarboxylic acid species measured.

In yet an even further embodiment, the amount of at least one long dicarboxylic acid species is a concentration.

In still yet an even further embodiment, the determined amount of the at least one long dicarboxylic acid species of the individual is greater than a threshold. And the individual is determined to have a high risk of Alzheimer's disease based on the amount of the at least one long dicarboxylic acid species being greater than the threshold.

In still yet an even further embodiment, the at least one long dicarboxylic acid species is pimelic acid (C7), suberic acid (C8), azelaic acid (C9), sebacic acid (C10), an unsaturated C7, C8, C9 or C10 dicarboxylic acid species, or a substituted C7, C8, C9 or C10 dicarboxylic acid species.

In still yet an even further embodiment, the method further performs an assay on the biological sample to determine a relative amount of at least one short dicarboxylic acid species in the sample. And the method determines a ratio of the relative amount of at least one long dicarboxylic acid species to the relative amount of at least one short dicarboxylic acid species. The determined ratio indicates the individual's risk of Alzheimer's disease.

In still yet an even further embodiment, the determined ratio of the individual is greater than a threshold, and wherein the individual is determined to have a high risk of Alzheimer's disease based on the ratio being greater than the threshold.

In still yet an even further embodiment, the threshold is based on the ratio of the concentration of at least one long dicarboxylic acid species to the concentration of at least one short dicarboxylic acid species in a cognitively healthy population or in a population of individuals having Alzheimer's disease.

In still yet an even further embodiment, the at least one short dicarboxylic acid specie is succinic acid (C4), glutaric acid (C5), an unsaturated C4 or C5 dicarboxylic acid specie, or a substituted C4 or C5 dicarboxylic acid specie.

In still yet an even further embodiment, the method further obtains at least a second biological sample of the individual. Each of the obtained biological samples contain dicarboxylic acids and at least two biological samples were acquired two different time points. The method adds an internal standard of dicarboxylic acid molecules to each biological sample. And the method performs an assay on each of the biological samples to determine concentrations of at least one long dicarboxylic acid species. The temporal change of the concentration of the at least one long dicarboxylic acid specie indicates the individual's risk of Alzheimer's disease.

In still yet an even further embodiment, an increase of the concentration of the long dicarboxylic acid species over time indicates a high risk of Alzheimer's disease.

In still yet an even further embodiment, the increase of the concentration of the long dicarboxylic acid species over time is greater than a threshold, indicating the high risk of Alzheimer's disease.

In still yet an even further embodiment, the threshold is based on the increase of the concentration of the long dicarboxylic acid species over time in a cognitively healthy population or in a population of individuals having Alzheimer's disease.

In still yet an even further embodiment, the method further performs an assay on the biological samples to determine a concentration of at least one short dicarboxylic acid species in each sample. And the method determines a ratio of the concentration of at least one long dicarboxylic acid species to the concentration of at least one short dicarboxylic acid species at each time point. The temporal change of the determined ratios indicates the individual's risk of Alzheimer's disease.

In still yet an even further embodiment, an increase of the concentration of at least one long dicarboxylic acid species to the concentration of at least one short dicarboxylic acid species over time indicates a high risk of Alzheimer's disease.

In still yet an even further embodiment, the increase of the ratio of the concentration of at least one long dicarboxylic acid species to the concentration of at least one short dicarboxylic acid species over time is greater than a threshold, indicating the high risk of Alzheimer's disease.

In still yet an even further embodiment, the threshold is based on the increase of the ratio of the concentration of at least one long dicarboxylic acid species to the concentration of at least one short dicarboxylic acid species over time in a cognitively healthy population or in a population of individuals having Alzheimer's disease.

In still yet an even further embodiment, the method further determines that the individual is at a high risk of Alzheimer's disease. And the method administers a diagnostic test to further assess the individual for Alzheimer's disease.

In still yet an even further embodiment, the diagnostic test is a cognitive test, a neuropsychological test, or medical imaging.

In still yet an even further embodiment, the diagnostic test is the Mini Mental State Exam or the Montreal Cognitive Assessment.

In still yet an even further embodiment, the method further determines that the individual is at a high risk of Alzheimer's disease. And the method administers a cognitive exercise to the individual for Alzheimer's disease.

In still yet an even further embodiment, the cognitive exercise is an activity that utilizes at least one of memory, reasoning, or information processing.

In still yet an even further embodiment, the method further determines that the individual is at a high risk of Alzheimer's disease. And the method administers a medication to the individual for Alzheimer's disease.

In still yet an even further embodiment, the medication is a cholinesterase inhibitor or a N-methyl D-aspartate receptor agonist.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1A illustrates a process for treating an individual based on their AD risk derived from dicarboxylic acid measurement data in accordance with an embodiment of the invention.

FIG. 1B illustrates a process for determining relative dicarboxylic acid concentrations in accordance with an embodiment of the invention.

FIG. 2 provides a pie graph detailing the average proportion of DCA in urine of a healthy individual, utilized in accordance with various embodiments of the invention.

FIG. 3 provides a bar graph detailing the differences of various DCA species between Alzheimer's disease patients (AD) and healthy controls (CH), utilized in accordance with various embodiments of the invention.

FIGS. 4A and 4B each provide a dot plot detailing the differences of various DCA species between AD patients, healthy controls with pathological amyloid/tau (CH-PAT), and healthy controls with normal amyloid/tau (CH-NAT), utilized in accordance with various embodiments of the invention.

FIG. 5 provides charts that compare short DCA species (C4+C5) and long DCA species (C7+C8+C9) in AD patients, healthy controls with pathological amyloid/tau (CH-PAT), and healthy controls with normal amyloid/tau (CH-NAT), utilized in accordance with various embodiments of the invention.

FIG. 6 provides ROC curves that show the specificity and sensitivity of distinguishing healthy controls with pathological amyloid/tau (CH-PAT), and healthy controls with normal amyloid/tau (CH-NAT), utilized in accordance with various embodiments of the invention.

FIGS. 7 through 11 each provide graphs depicting Spearman correlations of various DCA species with clinical covariates among AD patients and healthy controls, utilized in accordance with various embodiments of the invention.

FIG. 12 provides a schema explaining the correlations between various DCA species that distinguish AD patients, healthy controls with pathological amyloid/tau (CH-PAT), and healthy controls with normal amyloid/tau (CH-NAT), utilized in accordance with various embodiments of the invention.

FIG. 13 provides spectral depiction of various DCA species as determined by gas chromatography with mass spectrometry in accordance with an embodiment of the invention.

### DETAILED DESCRIPTION

Turning now to the drawings and data, methods and processes to assess and treat individuals based on their risk of Alzheimer's disease (AD) and applications thereof are described, in accordance with various embodiments of the invention. In several embodiments, analyte measurements of an individual are collected and used to determine an individual's AD risk. In some embodiments, lipid metabolites are used to determine risk of AD; in some particular embodiments dicarboxylic acids (DCAs) are used to determine AD risk. Many embodiments utilize an individual's AD risk determination to perform further diagnostics or a treatment upon that individual. In some instances, a diagnostic to be performed is a cognitive test, a neuropsychological test, medical imaging, or any combination thereof. In some instances, a treatment to be performed can include a medication, a dietary supplement, cognitive exercise, and any combination thereof.

Several embodiments utilize relative concentrations of DCAs to assess an individual's risk of AD. It should be understood that DCAs are to include unsaturated and/or substituted DCAs. Based on recent research findings, it is now understood that various DCAs are either increased or decreased in urinary excretion as AD develops. Furthermore, the changes of DCA constituency are able to be detected early, well before cognitive decline begins. Based on these findings, in some embodiments a relative decrease in succinic acid (C4) and/or glutaric acid (C5) is indicative of AD pathology. In a similar manner, in some embodiments a relative increase in pimelic acid (C7), suberic (C8) and/or azelaic acid (C9) is indicative of AD pathology. And in some embodiments, a decreasing amount of short DCAs (C4+C5) and/or an increasing amount of long DCAs (C7+C8+C9) is indicative of AD pathology. In some embodiments, relative ratios of DCAs are utilized to determine AD risk.

### Analytes Indicative of AD Risk

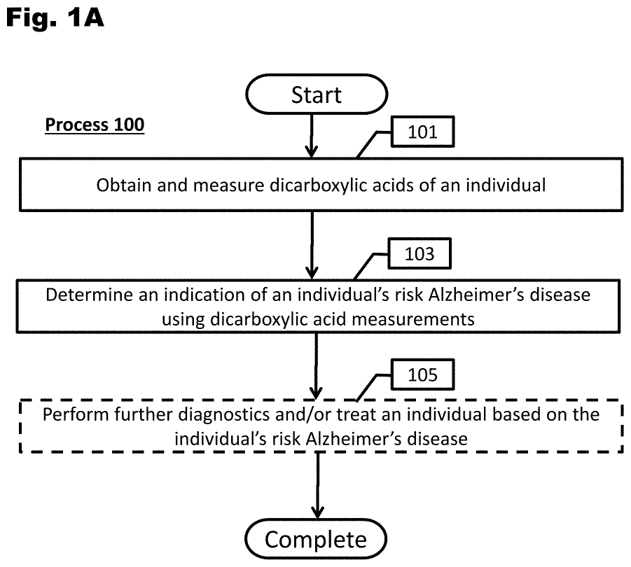

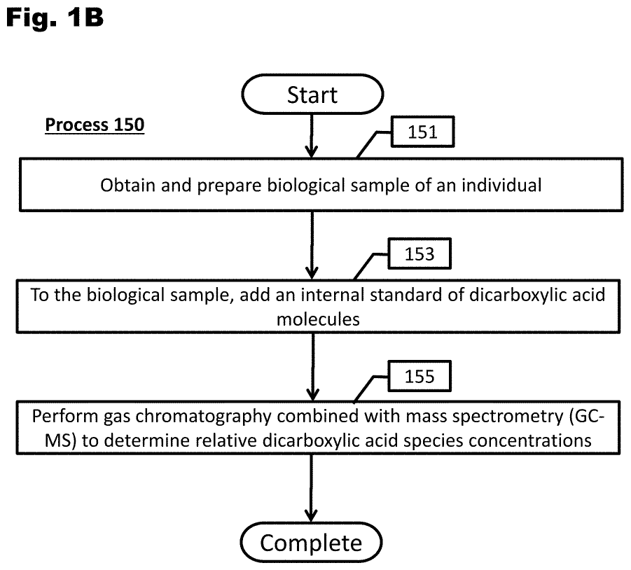

A process for determining an individual's AD risk using analyte measurements, in accordance with an embodiment of the invention is shown in FIG. 1A. This embodiment is directed to determining an individual's relative concentration of DCAs. In some embodiments, the knowledge garnered is utilized to perform further diagnostics and/or treat an individual. For example, this process can be used to identify an individual having a particular DCA constituency that is indicative of AD risk and treat that individual with a medication, a dietary supplement, cognitive exercise, or any combination thereof.

In a number of embodiments, analytes to be measure are lipid metabolites, and in particular DCAs. There are a number of DCAs that are metabolized and excreted in urine, including succinic acid (C4), glutaric acid (C5), adipic acid (C6), pimelic acid (C7), suberic acid (C8), azelaic acid (C9), sebacic acid (C10), unsaturated DCAs and substituted DCAs. An unsaturated DCA is one that has at least one carbon-carbon double bond and includes (but is not limited to) maleic acid, fumaric acid, gluconic acid, traumatic acid, muconic acid, glutinic acid, citraconic acid, mesconic acid, and itaconic acid. A substituted DCA is one having an organic group attached thereon, including (but not limited to) hydroxy, oxo and amino substituents. Examples of substituted DCAs include (but are not limited to) tartronic acid, mesoxalic acid, malic acid, tartaric acid, oxaloacetic acid, acetonedicarboxylic acid, α-hydroxyglutaric acid, α-ketoglutaric acid, diaminopimelic acid, and saccharic acid. It is now known that a relative concentration of DCAs indicate AD pathology, even at early stages before cognitive decline begins. Accordingly, measurements of a panel of DCAs, including unsaturated and substituted DCAs, can be used to assess an individual for AD risk. In some embodiments, analyte measures are used in lieu of standard AD diagnostic tests. In various embodiments, analyte measures are used to determine whether an individual should be further assessed for AD with a subsequent diagnostic test, such as neurological tests and medical imaging.

Process 100 begins with obtaining and measuring (101) analytes, such as DCAs, from an individual. In many instances, analytes are measured from a urine sample, but in some instances other sources could be used such as blood extraction, stool sample, or biopsy. In some embodiments, an individual's analytes are extracted during fasting, or in a controlled clinical assessment. A number of methods are known to extract analytes from an individual and can be used within various embodiments of the invention. In several embodiments, analytes are extracted over a period a time and measured at each time point, resulting in a dynamic analysis of the analytes. In some of these embodiments, analytes are measured with periodicity (e.g., monthly, quarterly, yearly).

In a number of embodiments, an individual is any individual that has their analytes extracted and measured. In some embodiments, an individual has not been diagnosed as having AD or at risk of developing AD. In some of these embodiments, the individual is cognitively healthy or diagnosed as cognitively healthy as determined by classical AD testing, including (but not limited to) neurological tests and medical imaging. In some of these embodiments, the individual has mild dementia or diagnosed with mild dementia as determined by classical AD testing, including (but not limited to) neurological tests and medical imaging. In a number of these embodiments, AD assessment is determined by standards recognized by an AD organization such as the guidelines provided by the National Institute of Aging (NIA). It should be understood that any well-respected AD organization guidelines used for diagnosis can be utilized in accordance with various embodiments of the invention.

In several embodiments, analytes to be used to indicate AD risk include (but not limited to) lipids, and especially DCAs. DCAs can be detected and measured by a number of methods, including chromatography and mass spectrometry, especially gas chromatography with mass spectrometry (GC-MS). In several embodiments, an internal standard is added to the sample containing DCA to perform measurements. In some embodiments, the standards are deuterated DCAs having a known concentration.

In several embodiments, DCA measurements are performed by taking a single time-point measurement. In many embodiments, DCA measurements are performed by taking multiple time-point measurements over a period of time, which provides the change (increase or decrease) of DCAs over time. Various embodiments incorporate correlations, which can be calculated by a number of methods, such as the Spearman correlation method. A number of embodiments utilize a computational model that incorporates analyte measurements, such as linear regression models. Significance can be determined by calculating p-values that are corrected for multiple hypothesis. It should be noted however, that there are several correlation, computational models, and statistical methods that can utilize analyte measurements and may also fall within some embodiments of the invention.

Using measurements of DCAs, process 100 determines (103) an indication of an individual's AD risk. In many embodiments, the correlations and/or computational models can be used to indicate a result of AD risk. In several embodiments, determining analyte correlations or modeling AD risk is used for early detection. In various embodiments, measurements of analytes can be used as a precursor indicator to determine whether to perform a further diagnostic.

Based on studies performed, it has been found that several DCA measurements correlate with AD pathology and thus can serve as surrogates to determine AD risk. Correlative DCAs include (but are not limited to) succinic acid (C4), glutaric acid (C5), pimelic acid (C7), suberic (C8), azelaic acid (C9), combination of short DCAs (C4+C5), and/or combination of long DCAs (C7+C8+C9+C10). In some embodiments, a decrease of succinic acid (C4) and/or glutaric acid (C5) over time is indicative of a high risk of AD. In a similar manner, in some embodiments an increase of pimelic acid (C7), suberic (C8) and/or azelaic acid (C9) over time is indicative of a high risk of AD. In some embodiments, a decreasing amount of one or more short DCA species (C4+C5) and/or an increasing amount of one or more long DCA species (C7+C8+C9+C10) over time is indicative of a high risk of AD. Short and/or long DCAs can be combined in any appropriate way, including (but limited to) summed, averaged, and weighted average.

Further, DCAs measurements can be concentrations of DCAs or relative amounts of DCAs. A relative amount of a DCA can be relative to a set of one or more DCAs measured. In some instances, each DCA measurement is the amount of the particular DCA to the total amount of DCAs measured.

In some embodiments, the ratio of long DCAs to short DCAs is analyzed, which can be done in a variety of ways. In some embodiments, a high ratio of long DCAs (C7+C8+C9+Cl 0) to short DCAs (C4+C5) is indicative of a high risk of AD. Alternatively, a low ratio of short DCAs (C4+C5) to long DCAs (C7+C8+C9+C10) is indicative of a high risk of AD. Likewise, in some embodiments, an increase of the ratio of long DCAs (C7+C8+C9+C10) to short DCAs (C4+C5) over time, and vice versa, is indicative of a high risk of AD. It should be understood that any ratio between short and long DCAs could be utilized. Accordingly, various embodiments utilize ratios of C4 and/or C5 (alone or in combination) to C7 and/or C8 and/or C9 and/or C10 (alone or in any combination).

In some embodiments, a threshold is utilized to determine whether a DCA measurement or ratio is indicative of a high risk of AD. For instance, in some embodiments, an amount of one or more long DCA species (C7+C8+C9+Cl 0) greater than a threshold indicates high risk of AD. Likewise, in some embodiments, an amount of one or more short DCA species (C4+C5) less than a threshold indicates high risk of AD. In some embodiments, an increase of the amount of one or more long DCA species (C7+C8+C9+C10) over time greater than threshold indicates a high risk of AD. In some embodiments, a decrease of the amount of one or more short DCA species (C4+C5) over time less than threshold indicates a high risk of AD. In some embodiments, a high ratio of long DCAs (C7+C8+C9+C10) to short DCAs (C4+C5) greater than threshold indicates a high risk of AD. Alternatively, a low ratio of short DCAs (C4+C5) to long DCAs (C7+C8+C9+C10) less than a threshold indicates of a high risk of AD. Likewise, in some embodiments, an increase of the ratio of long DCAs (C7+C8+C9+Cl 0) to short DCAs (C4+C5) over time greater than a threshold, and vice versa, indicates a of a high risk of AD. A threshold can be determined by any appropriate means. In various embodiments, a threshold is determined by DCA measurements and ratios of a population of cognitively healthy individual, individuals having AD, or any combination thereof.

Having determined an individual's AD risk, further diagnostics or a treatment can optionally be performed on the individual (105). In a number of embodiments, a diagnostic to be performed is a cognitive test, a neuropsychological test, medical imaging or any combination thereof. In a number of embodiments, a treatment to be performed entails a medication, a dietary supplement, cognitive exercise, or any combination thereof. In some embodiments, an individual is treated by medical professional, such as a doctor, nurse, dietician, or similar. Various embodiments are directed to self-treatment such that an individual having a particular AD risk intake a medicine, a dietary supplement, alters her diet, or cognitively exercises based on the knowledge of her indicated AD risk.

While specific examples of determining an individual's AD risk are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for determining an individual's AD risk appropriate to the requirements of a given application can be utilized in accordance with various embodiments of the invention.

### Methods of Measuring Analytes of AD Risk

In several embodiments, biomarkers are detected and measured, and based on the relative amount of the biomarker, AD risk can be determined. Biomarkers that can be used in the practice of the invention include (but are not limited to) lipids, and especially DCAs. Correlative DCAs include (but are not limited to) succinic acid (C4), glutaric acid (C5), pimelic acid (C7), suberic (C8), azelaic acid (C9), combination of C4+C5, and/or combination of C7+C8+C9. It is noted, in some embodiments, a combination of C7+C8+C9+C10 may be utilized instead of C7+C8+C9.

### Detecting and Measuring Levels of Biomarkers

Analyte biomarkers in a biological sample (e.g., urine sample) can be determined by a number of suitable methods. Suitable methods include chromatography (e.g., high-performance liquid chromatography (HPLC), gas chromatography (GC), liquid chromatography (LC)), mass spectrometry (e.g., MS, MS-MS), NMR, enzymatic or biochemical reactions, immunoassay, and combinations thereof. For example, mass spectrometry can be combined with chromatographic methods, such as liquid chromatography (LC), gas chromatography (GC), or electrophoresis to separate the metabolite being measured from other components in the biological sample. See, e.g., Hyotylainen (2012) Expert Rev. Mol. Diagn. 12(5):527-538; Beckonert et al. (2007) Nat. Protoc. 2(11):2692-2703; O'Connell (2012) Bioanalysis 4(4):431-451; and Eckhart et al. (2012) Clin. Transl. Sci. 5(3):285-288; the disclosures of which are herein incorporated by reference. Alternatively, analytes can be measured with biochemical or enzymatic assays. In another example, biomarkers can be separated by chromatography and relative levels of a biomarker can be determined from analysis of a chromatogram by integration of the peak area for the eluted biomarker.

The methods for detecting biomarkers in a sample have many applications. For example, the biomarkers are useful in monitoring individuals as they age. In several embodiments, methods to detect DCAs are performed prior to an individual displaying signs of cognitive decline, which can help with early detection and early treatment options.

Gas chromatography combined with mass spectrometry

Provided in FIG. 1B is a method determine relative concentrations of DCA constituents utilizing gas chromatography combined with mass spectrometry (GC-MS). Process 150 begins with obtaining and preparing (151) a biological sample of an individual to be examined. A biological sample can include any sample containing DCA constituents, including a urine sample, blood draw, cerebrospinal fluid draw, stool sample, or a tissue biopsy. In several embodiments, a urine sample is utilized for ease of acquisition.

Once a biological is obtained, it can be prepared for analysis. Debris and cells in the biological sample can be removed by any appropriate method, such as (for example) centrifugation. In addition, the sample can be diluted and/or concentrated to an appropriate degree. Various analysis can be performed on the biological sample to standardize and ensure the sample meets appropriate standards. For example, in some embodiments, a urine sample can be diluted 10- to 20-fold and various proteins (e.g., creatinine, albumin) are utilized to standardize the biological samples.

Process 150 also adds (153) an internal standard of DCA molecules to the biological sample. In some embodiments, a deuterated standard of DCA molecules are utilized, which can be obtained from various vendors such as Cambridge Isotope Laboratory (Tewksbury, Mass.). Having an internal standard mixed within, the biological samples can be prepared for chromatography and spectrometry. In some embodiments, DCA molecules (including the deuterated DCA standards) are converted to dipentafluorobenzyl esters prior to GC-MS analysis. For a detailed explanation of preparing DCA molecules for GC-MS analysis, see the “Dicarboxylic acid extraction and derivatization” section within the Exemplary Embodiments.

Process 150 further performs (155) GC-MS to determine relative DCA concentrations. DCAs have two reactive carboxylic acid groups, allowing for the detection of the parent mass M+2PFB. [M+1PFB]⁻carboxylate ions (m/z). Utilization of GC-MS to determine relative DCA concentrations has proven to be reliable and reproducible (See Exemplary Embodiments for data).

### Biochemical and Enzymatic Assays

Various embodiments are directed towards chromogenic, chemiluminescent and/or fluorescent methods to detect DCAs in a sample. Accordingly, a biochemical or enzymatic assay is performed to yield a chromogenic, chemiluminescent or fluorescent response indicative relative DCA amount. In some embodiments, a chromogenic, chemiluminescent or fluorescent assay is able to detect and differentiate short DCAs (e.g., succinic acid (C4) and glutaric acid (C5)) from long DCAs (e.g., pimelic acid (C7), suberic (C8), azelaic acid (C9), and sebacic acid (C10)). In some embodiments, a chromogenic, chemiluminescent or fluorescent assay is able to detect and differentiate at least one DCA from all other DCAs.

### Immunological Detection of DCAs

A number of embodiments are directed towards the use of antibodies to detect DCAs in a sample. Accordingly, antibodies specific for various DCAs can be utilized to determine a relative of a DCA species in a sample. In some embodiments, an immunoassay is able to detect and differentiate short DCAs (e.g., succinic acid (C4) and glutaric acid (C5)) from long DCAs (e.g., pimelic acid (C7), suberic (C8), azelaic acid (C9), and sebacic acid (C10)). In some embodiments, an immunoassay is able to detect and differentiate at least one DCA from all other DCAs.

Immunoassays based on the use of antibodies that specifically recognize a DCAs may be used for measurement of DCA levels. Such assays include (but are not limited to) enzyme-linked immunosorbent assay (ELISA), radioimmunoassays (RIA), “sandwich” immunoassays, fluorescent immunoassays, enzyme multiplied immunoassay technique (EMIT), capillary electrophoresis immunoassays (CEIA), immunoprecipitation assays, western blotting, immunohistochemistry (IHC), flow cytometry, and cytometry by time of flight (CyTOF).

Antibodies that specifically bind to a DCA can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A DCA antigen can be used to immunize a mammal, such as a mouse, rat, rabbit, guinea pig, monkey, or human, to produce polyclonal antibodies. If desired, a DCA antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface-active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a DCA antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 31 42, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobulin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prev. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J Cancer 61, 497-501, 1995; Nicholls et al., J. Immunol. Meth. 165, 81-91, 1993).

Antibodies which specifically bind to a DCA antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant DCA is bound. The bound antibodies can then be eluted from the column using a buffer with a high salt concentration.

Antibodies may be used in diagnostic assays to detect the presence or for quantification of DCA in a biological sample. Such a diagnostic assay may comprise at least two steps; (i) contacting a biological sample with the antibody, and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 1251, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem. 13:1014 (1974); Pain et al., J. Immunol. Methods 40:219 (1981); and Nygren, J. Histochem. and Cytochem. 30:407 (1982).

Immunoassays can be used to determine the presence or absence of a DCA in a sample as well as the quantity of a DCA in a sample. First, a test amount of a DCA in a sample can be detected using the immunoassay methods described above. If a DCA is present in the sample, it will form an antibody-biomarker complex with an antibody that specifically binds the DCA under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

### Kits

In several embodiments, kits are utilized for monitoring individuals for AD risk, wherein the kits can be used to detect DCA biomarkers as described herein. For example, the kits can be used to detect any one or more of the DCA biomarkers described herein, which can be used to determine AD risk. The kit may include one or more agents for detection of one or more biomarkers, a container for holding a biological sample (e.g., urine) obtained from a subject; and printed instructions for preparing agents with the biological sample to detect the presence or amount of one or more biomarkers in the sample. The agents may be packaged in separate containers. The kit may further comprise one or more control reference samples and reagents for performing a biochemical assay, enzymatic assay, immunoassay, or chromatography. In some embodiments, the kit may include deuterated DCA standards and/or reagents to prepare a sample for GC-MS analysis (e.g., hydrochloric acid, ethyl acetate, sodium sulfate, pentafluorobenzyl bromide (PFBBr), and diisopropylethylamine (DIPEA)). In some embodiments, a kit may contain reagents for performing chromatography (e.g., resin, solvent, and/or column).

A kit can include one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of determining DCA concentrations in a sample.

Applications and Treatments Related to AD risk

Various embodiments are directed to diagnostics and treatments related to AD risk. As described herein, an individual may have their AD risk indicated by various methods. Based on one's AD risk indication, an individual can be subjected to further diagnostics and/or treated with various medications, dietary supplements, and cognitive exercise regimens.

### Clinical Diagnostics

A number of embodiments are directed towards diagnosing individuals using relative amount of DCA constituents in their biological samples. In some embodiments, correlation methods or a trained computational model produces an AD risk score indicative of likelihood to develop AD.

In a number of embodiments, diagnostics can be performed as follows:
a) obtain DCA measurement data of the individual to be diagnosed
b) determine AD risk score
c) diagnose the individual based on the AD risk score.

Diagnoses, in accordance with various embodiments, can be performed as portrayed and described in herein, such as portrayed in FIG. 1.

### Diagnostics, Medications and Supplements

Several embodiments are directed to the use of medications and/or dietary supplements to treat an individual based on having a high risk of AD. In some embodiments, medications and/or dietary supplements are administered in a therapeutically effective amount as part of a course of treatment. As used in this context, to “treat” means to ameliorate at least one symptom of the disorder to be treated or to provide a beneficial physiological effect. A therapeutically effective amount can be an amount sufficient to prevent reduce, ameliorate or eliminate symptoms of AD and/or reduce the risk of AD. For example, a therapeutically effective amount can be an amount to improve cognition and/or prevent cognitive decline. Alternatively, a therapeutically effective amount can be an amount to reduce loss of brain matter.

Dosage, toxicity and therapeutic efficacy of the compounds can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to other tissue and organs and, thereby, reduce side effects.

Data obtained from cell culture assays or animal studies can be used in formulating a range of dosage for use in humans. If the pharmaceutical is provided systemically, the dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of AD progression) as determined by an appropriate means (e.g., amyloid and/or tau accumulation). Such information can be used to more accurately determine useful doses in humans. Levels in plasma may be measured, for example, by liquid chromatography coupled to mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result.

A number of diagnostic tests are available to further assess AD. Diagnostic tests include (but are not limited to) cognitive tests, neuropsychological tests, and medical imaging. Cognitive tests may be applied to test the individual's ability memory and cognition. Neuropsychological tests may be administered to determine if the individual has dementia and/or able to conduct daily tasks such as driving and/or managing finances. Cognitive and neuropsychological tests include (but are not limited to) Mini Mental State Exam (MMSE) and the Montreal Cognitive Assessment (MoCA) (www.mocatest.org). Many medical imaging techniques can be performed, including magnetic resonance imaging (MRI), computerized tomography (CT), and positron emission tomography. MRIs and CTs can be utilized to detect brain matter loss, especially in the hippocampus. PET scans can be utilized to detect areas of degeneration, amyloid plaques, and/or tau neurofibrillary tangles.

A number of medications are available to treat AD. Medications include (but are not limited to) cholinesterase inhibitors (e.g., donepezil, galantamine, rivastigmine, and tacrine), and N-methyl D-aspartate (NMDA) receptor agonists (e.g., memantine). Accordingly, an individual may be treated, in accordance with various embodiments, by a single medication or a combination of medications described herein. Furthermore, several embodiments of treatments further incorporate dietary supplements (e.g., antioxidants, resveratrol, vitamin D and ginkgo Biloba).

A number of cognitive exercises can also be performed to help treat individuals with risk of developing AD. In general, a cognitive exercise is an activity that utilizes at least one of memory, reasoning, or information processing. In some embodiments, an individual with risk of developing AD takes on new learning opportunities, such as taking educational classes, learning a second language, or learning an instrument. In some embodiments, an individual with risk of developing AD play board games and puzzles (e.g., mahjong, Sudoku, and crossword). In some embodiments, an individual with risk of developing AD writes and/or orally recalls memoirs to help keep memory fresh.

### Exemplary Embodiments

Biological data support the methods and systems of assessing AD risk and applications thereof. In the ensuing sections, exemplary methods and exemplary applications related to analyte panels, correlations, and AD risk are provided.

As described in these examples, a goal of these studies was to develop non-invasive biomarkers to enable widespread screening and early diagnosis of Alzheimer's disease (AD). It was hypothesized that the loss of brain tissue in AD will result in detection of brain lipid components in urine, and that these will change in concert with CSF and brain biomarkers of AD. In particular, dicarboxylic acids (DCA) were examined in urine, which may reflect products of oxidative damage and energy generation/balance that may account for changes in brain function in AD.

The DCA excretion hypothesis is based on the following. DCAs are formed from the oxidative breakdown of unsaturated fatty acids and the increase in oxidative stress associated with AD is predicted to alter DCA formation from long chain monounsaturated and polyunsaturated fatty acids. Several DCAs such as succinic acid and glutaric acid contribute to energy metabolism and changes in their levels may impact mitochondrial function. Mitochondrial function and energy imbalance are proposed to contribute to AD pathology. DCAs are known to inhibit mitochondrial ATP production and alter respiration. Moreover, modification of several mitochondrial proteins by succinylation is suggested to impose dysfunctional consequence. Thus, oxidative stress will manifest in the urinary excretion of DCAs. In sum, the dysfunctional brain mitochondria as reported in AD may account for the reduction some DCAs, which in turn leads to oxidative damage of brain lipids and results in the loss of brain tissue and urinary excretion of oxidized DCAs products.

In these examples, urine was examined from individuals that were selected at higher risk of AD because of their age, and classified them as cognitively healthy (CH) after an extensive neuropsychometric battery and the Uniform Data Set-2 criteria of the National Alzheimer's Coordinating Centers (NACC). Based on a previous report that demonstrated the logistic regression from CSF amyloid and Tau levels correctly classify individuals with clinically probable AD, these regression analyses were used this to distinguish age-matched CH individuals with normal amyloid/tau (CH-NAT) or pathological amyloid/tau (CH-PAT) (See M. G. Harrington, et al., PloS one 8, e79378 (2013), the disclosure of which is herein incorporated by reference). In a four-year follow-up, none of the CH-NATs but 40% of the CH-PATs declined cognitively.

The data provided herein show that C4-05 DCAs decreased and C7-C10 DCAs increased in the urine from AD compared to CH individuals. The results, which are detailed in the ensuing sections, showed short chain DCAs positively correlated with CSF Aβ₄₂, while C7-C10 DCAs negatively correlated with CSF Aβ₄₂ and positively correlated with CSF Tau. A link between the changes in urine DCAs and brain pathology is further supported by finding a negative correlation of C7-C10 DCAs with hippocampal volumes (left: r=−0.47; p=0.0056, right: r=−0.49; p=0.0040, total: r=−0.48; p=0.0041), which was not found in other brain regions. These data provide that urine increased lipoxidation and measures of dysfunctional energy balance are hallmarks of early AD pathology. Routine measures of urine DCAs can contribute to personalized healthcare by indicating disease progression, and can be utilized to explore population wellness or monitor the efficacy of therapies in clinical trials.

### Research Results

Over 100 study participants>70 years were classified by NACC UDS-2 criteria and consensus conferencing as cognitively healthy (CH, n=76) or probable AD (n=24). Those with mild cognitive impairment were excluded to reduce heterogeneity in the analysis. CH individuals were sub-classified by CSF Aβ₄₂ and Tau into CH-NAT (n=45), or CH-PAT (n=31). The groups were of similar age, and women comprised 58.3-66.7% across the groups (Table 1.). These individuals were genotyped (when possible) to determine their ApoE status, and their BMI were compared and the number of years of education were averaged. In the latter case, AD individuals had less formal education than CH (p=0.036), typical for AD.

In order to account for kidney function and hydration levels, the urine concentrations of total protein, creatinine, and albumin, and the urinary albumin to creatinine ratio (UACR) were analyzed. Individuals with AD showed evidence of kidney function impairment through higher concentrations of total protein, albumin, and UACR compared to controls (Table 1), consistent with the higher level of albuminuria recognized with cognitive decline.

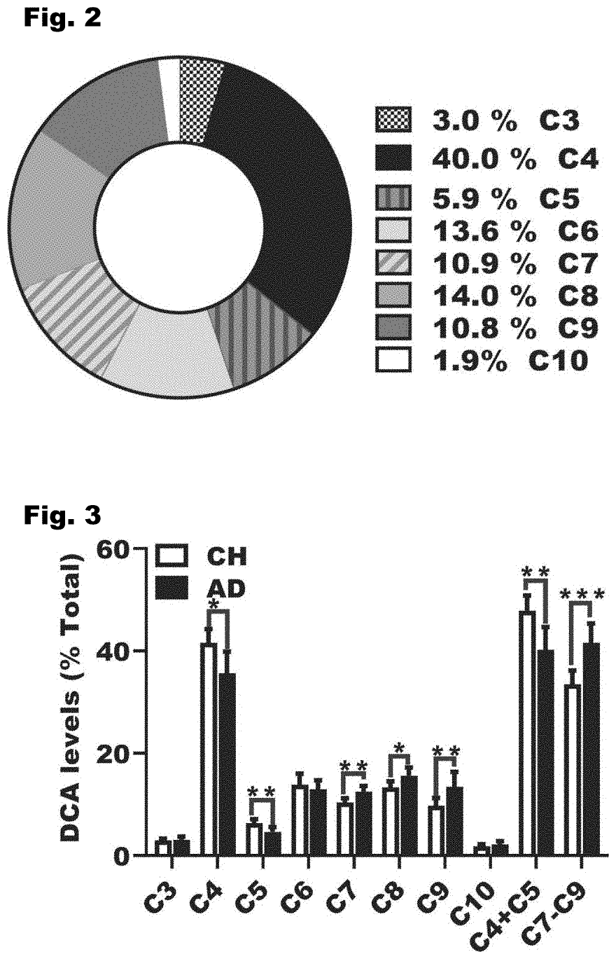

Detection of dicarboxylic acids in urine: Eight (8) DCAs in urine were quantified from cognitively healthy and AD individuals: malonic (C3), succinic (C4), glutaric (C5), adipic (C6), pimelic (C7), suberic (C8), azelaic (C9), and sebacic acids (C10). C4 accounted for with the majority of (42%, range 34.7%-44.1%) of DCAs detected in urine while C6, C8, C7, and C9 each represented>10% of total urine DCA (FIG. 2). C5, C3, and Cl 0 accounted 6%, 3% and 2% of total urine DCA, respectively.

Urine dicarboxylic acid species differ in CH compared with AD: The total amount (mean+standard deviation) of DCA species was 6.68±3.92 μg/mL and 7.86±4.54 μg/mL for CH and AD clinical groups, respectively. While there was no significant difference between the total amount of DCA species, for some individual acids mean levels were significantly higher in the AD group compared to the CH group (FIG. 3): pimelic, p=0.0033; suberic, p=0.0175; azelaic, p=0.0010; and sebacic acids, p=0.0051.

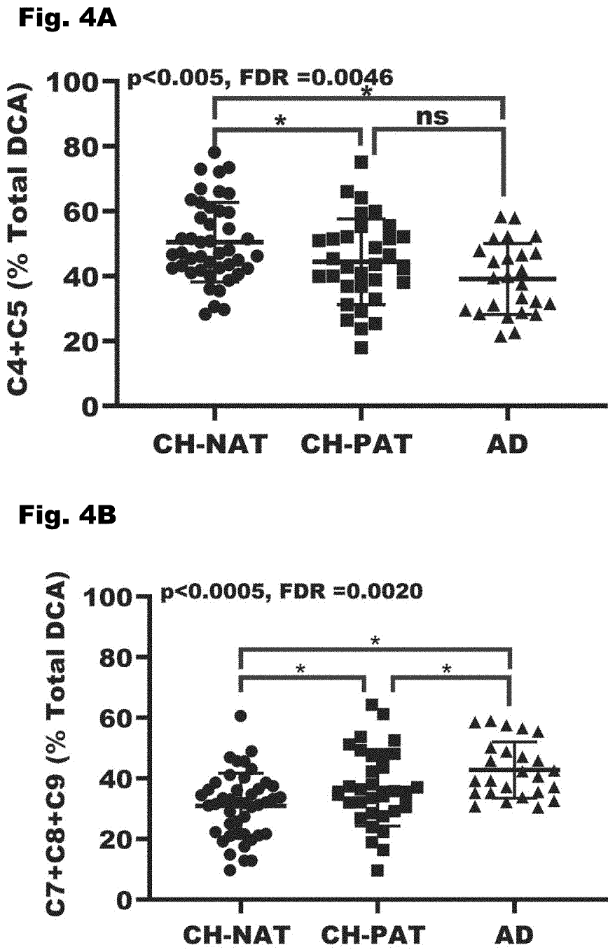

To normalize between urine samples, levels of individual DCA species were expressed as a percentage of total DCA species. Mean proportions of succinic (p=0.0113) and glutaric acids (p=0.0087) were significantly lower in AD compared to CH. On the other hand, mean proportions of pimelic (p=0.0035), suberic (p=0.0161), and azelaic acids (p=0.0022) were significantly higher in AD compared to CH (FIG. 4A). The accuracy of the clinical group classification was enhanced when we combined the sum of metabolic process DCAs and the sum of oxidized products of longer chain fatty acids, as illustrated by lower p values (sum of C4 and C5: p=0.0059; sum of C7 through C9: p=0.0004), FIG. 4B.

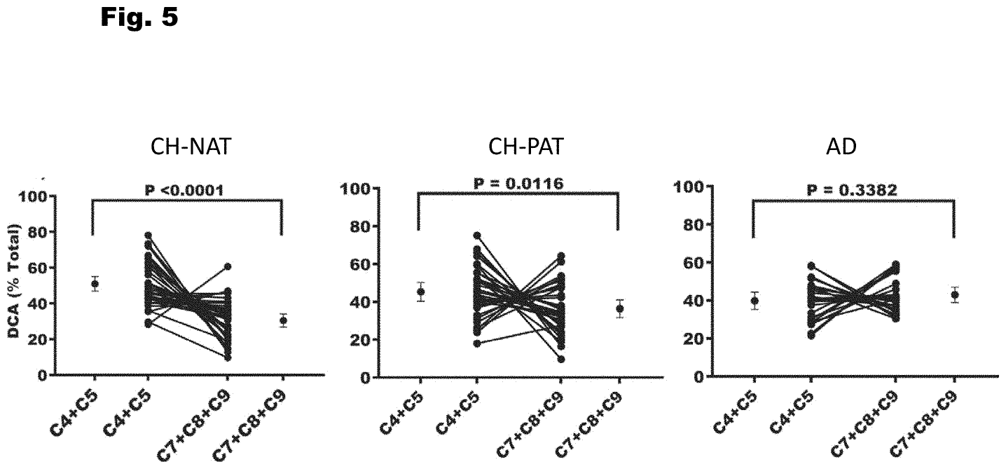

When the CH group was further sub-classified based on CSF amyloid and total tau to distinguish those CH individuals at higher risk of developing AD, the differences in DCA species between CH-NAT, CH-PAT, and AD were identifiable. Examination showed that the DCA group that was higher in AD is mainly derived from the breakdown of unsaturated fatty acids while the DCA group that was lower in AD is composed of components of the TCA cycle (FIG. 5).

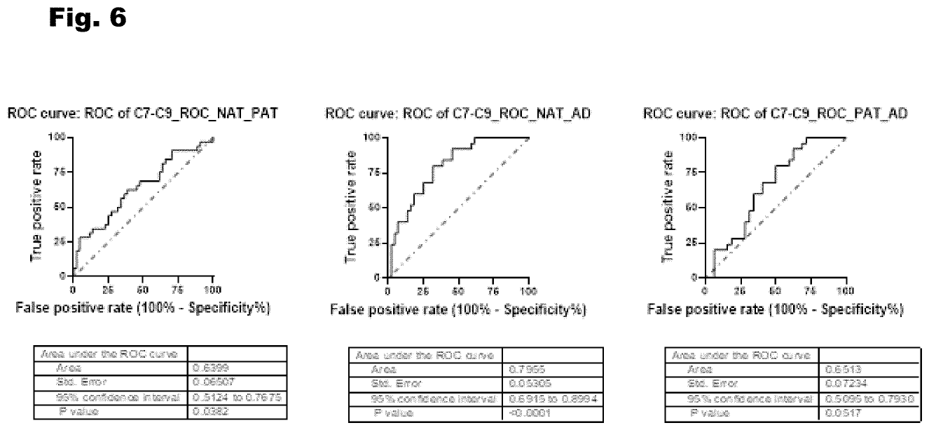

The sensitivity and specificity between these three clinical groups to differentiate C7 through C9 is depicted in the receiver operating characteristics (ROC) curves in FIG. 6.

Multivariable analysis of urinary DCA changes for C4/C5 and adjustment for multiplicity: Of the candidate confounders age, sex, smoking status, and Stroop Interference score, only smoking status was close to being a significant independent predictor of C4/C5 (p=0.07). With smoking status included as a covariate and using the Tukey-Kramer adjustment for multiplicity, there was a significant difference between CH-NATs and CH-PATs (p=0.04), and between CH-NATs and AD (p=0.0004), but not between CH-PATs and AD (p=0.26).

Multivariable analysis of urinary DCA changes for C7-C9 and adjustment for multiplicity: For C7-C9, only age was close to being a significant independent predictor (p=0.10). With age included as a covariate and using the Tukey-Kramer adjustment for multiplicity, the comparison between CH-NATs and AD was highly significant (p=0.0002) whereas the comparisons between CH-PATs and CH-NATs and between CH-PATs and AD were not significant (p=0.09 and 0.12, respectively).

Predictive ability of DCAs for clinical and CSF classification: A multinomial logistic model was developed and tested to predict membership to CH-NAT, CH-PAT, and AD groups based on C7-C9 DCAs. The model correctly predicted group for 46 of 101 (45.5%) individuals based on their C7_C9 values: 36 of 44 CH-NAT (82%) but only 2 of 32 CH-PAT (6%) and 8 of 25 AD (32%). Specificity for CH-NAT, CH-PAT, and AD was 42% (24/57), 86% (59/69), and 84% (64/76), respectively.

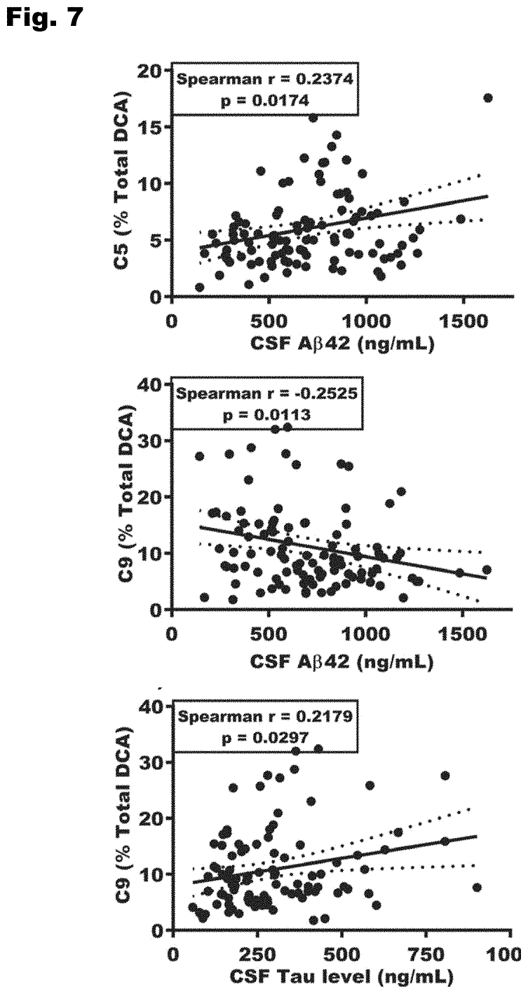

Urine DCAs correlate with CSF and MRI biomarkers of AD To determine if urinary DCA species relate to brain degeneration, their correlations with CSF Aβ₄₂ and Tau protein levels were examined. The scatter plots (FIG. 7) show that glutaric acid positively correlated with Aβ₄₂ (r=0.23; p=0.0186) while azelaic acid negatively correlated with Aβ₄₂ (r=−0.26; p=0.0101). Positive correlations were found with CSF Tau for azelaic (r=0.22, p=0.0276) (FIG. 7) and sebacic acids (r=0.20; p=0.0476) individually, and for the sum of C7-C10 (r=0.20; p=0.0499).

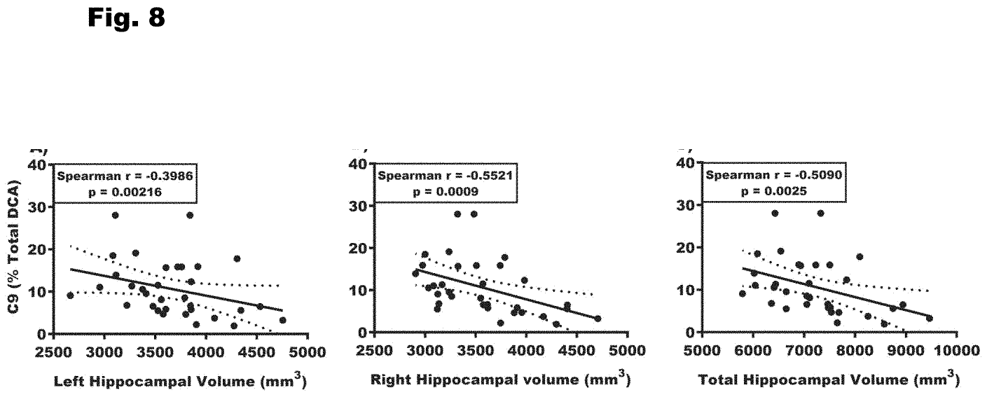

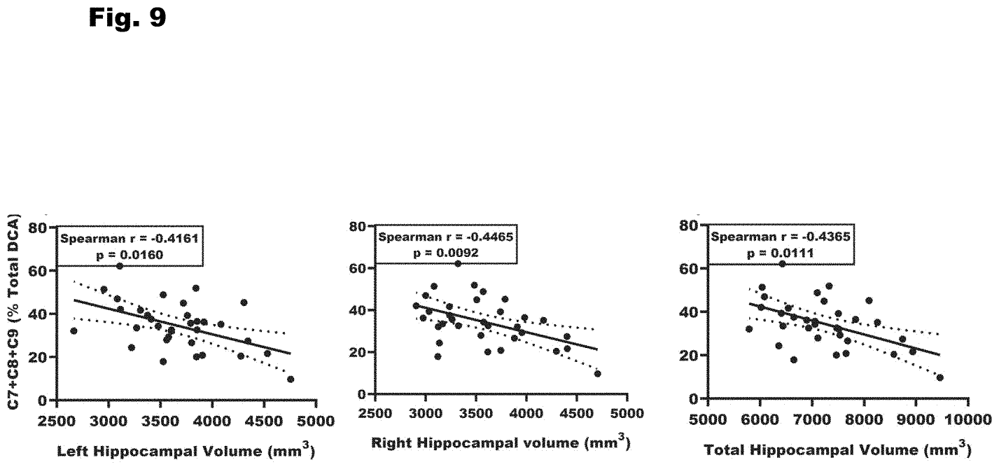

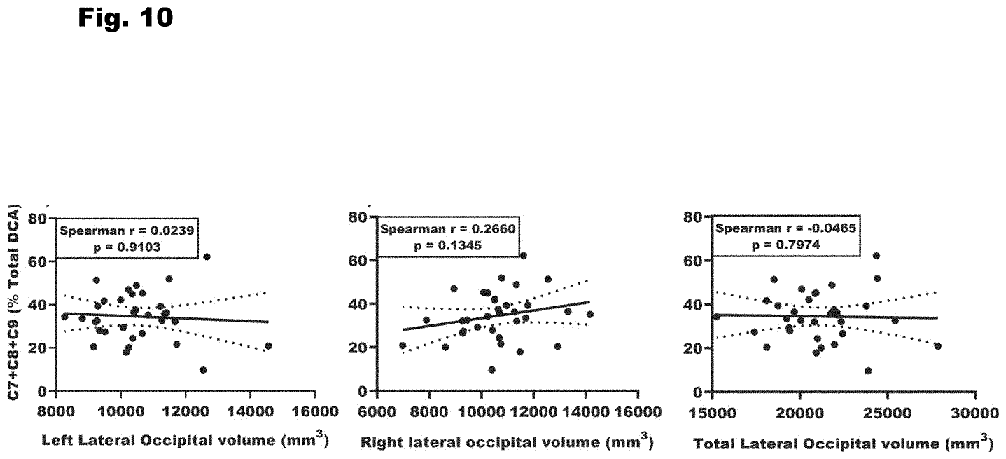

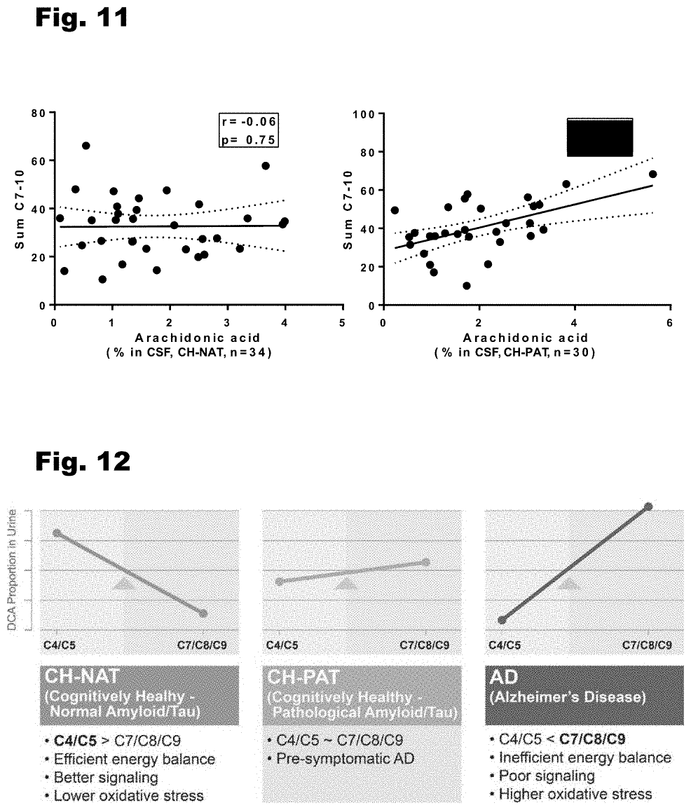

It was tested whether the breakdown species C7 through C10 could be linked to the hippocampal volume by magnetic resonance imaging (MRI). FIGS. 8, 9, and 10 show a negative correlation between the percentage of breakdown species and hippocampal volume (left: r=−0.47; p=0.0056, right: r=−0.49; p=0.0040, total: r=−0.48; p=0.0041,). In contrast, no correlation was found of the combined C7-9 DCAs with the lateral occipital lobe volume, selected as a control region that is marginally affected in Alzheimer's disease (FIG. 10). Importantly, measures of C7-C10 species associate with the changes in brain-derived CSF fatty acid precursors in the pre-symptomatic CH-PAT cohort (FIG. 11).

Biochemical and clinical implication of the interaction of changes of DCAs: The studies within these examples show diametrically opposed changes in two groups of DCAs in urine (FIG. 12). While energy-related C4/C5 are higher and oxidatively derived C7/C8/C9 are lower in cognitively healthy study participants, the opposite levels are present in the urine from AD participants. Functionally, these two groups of DCAs also have opposite effects. For example, succinate is a cofactor in energy metabolism via the TCA cycle while azelaic acid is known to inhibit several TCA enzymes and mitochondrial electron transport proteins. If the clearance of amyloid via autophagocytosis, the repair of post mitotic neurons, and other processes required for maintaining a healthy brain require energy, a higher C4/C5 and lower C7/C8/C9 is desirable. On the other hand, a lower C4/C5 and a higher C7/C8/C9 will favor the accumulation of amyloid, resulting in brain dysfunction that characterizes AD pathology. The implications of this study are that strategies that increase C4/C5 and decrease C7/C8/C9 can enhance cognitive function or diminish AD progression.

### Methods of Analysis

### Diagnosis of Study Participants

The Huntington Memorial Hospital Institution Review Board, Pasadena, Calif., approved the protocol and consent forms for this study. All study participants gave written, informed consent. Participants between 70 and 100 years of age were recruited from the greater Los Angeles area, and medical and neuropsychological diagnostic processes for this study have been previously described (See M. G. Harrington, et al., (2013), cited supra). Initially, the study participants were divided based on neuropsychological studies into 2 groups, cognitively normal (CH, n=76) and presumed AD (AD, n=24). The CH group was further divided into asymptomatic low risk individuals (CH-NAT, n=45), and asymptomatic high risk individuals (CH-PAT, n=31), based on beta amyloid₄₂/tau ratios in the cerebrospinal fluid (CSF) (See M. G. Harrington, et al., (2013), cited supra).

### Measures of Brain Volume by MRI

The MRI datasets were obtained using a GE 3 or 1.5T MR scanner with a standard eight-channel array head coil at HMRI. Anatomical coronal spin echo T2-weighted scans were first obtained through the hippocampi (TR/TE 1550/97.15 ms, NEX=1, slice thickness 5 mm with no gap, FOV=188×180 mm, matrix size=384×384). Baseline coronal T1-weighted maps were then acquired using a T1-weighted 3D fast spoiled gradient echo (FSPGR) pulse sequence and variable flip angle method using flip angles of 2°, 5° and 10°. Data was analyzed using Freesurfer 6.0 (Freesurfer, Harvard) to obtain hippocampal and occipital lobe volumes.

### Urine Collection, Total Protein, Albumin, and Creatinine

A single point mid-stream specimen of urine was collected from study participants after an overnight fast, between 8:00 am and 10:00 am. After centrifugation to remove any debris, urine was fractionated and stored in polycarbonate tubes at −80° C. until required for analyses. Urine was diluted (10-20×) and levels of creatinine determined using the improved Jaffe method using picrate using creatinine (0-15 mg/dL) as a standard (Creatinine kit, #500701, Cayman Chemical Company, Ann Arbor, Mich.). Urine albumin was quantified using size exclusion chromatography (HP1050) on a Zorbax GF-250 column (4.6×250 mm) using 0.1 PBS (pH 7.0) at a flow rate of 0.5 mL/min. The column was calibrated with thyroglobulin (670 kDa), gamma globulin (158 kDa), ovalbumin (44 kDa), myoglobulin (17 kDa), and vitamin B-12 (1.35 kDa) and levels of albumin calculated (mg/mL).

### Dicarboxylic Acid Extraction and Derivatization

The extraction protocol was adapted from Costa et al. (Journal of Pharmaceutical and Biomedical Analysis 21, 1215-1224 (2000), the disclosure of which is herein incorporated by reference). Briefly, 500 μL urine and 100 μL deuterated internal standard mixture at 20 ng/μL in ethanol was diluted to 1 mL with brine solution and acidified to pH 2 with 3 drops of 1 M HCl. Then, the urine was extracted 3 times with 3 mL ethyl acetate. The combined organic layer was dried with sodium sulfate before decanting and drying under a stream of nitrogen at 45° C. Once dry, the extracted DCA were converted to dipentafluorobenzyl esters by adding 25 μL of 5% v/v PFBBr and 25 μL 10% v/v DIPEA in anhydrous acetonitrile to the residue. The reaction was allowed to proceed for 30 min at 60° C. The reaction solution was then dried under a stream of nitrogen before adding 1 mL of hexanes to the reaction tube, vortexed for 10 min, and then transferred to GC/MS vials. After evaporation under a stream of N2, the derivatized residue was dissolved in 100 μL dodecane for GC/MS analysis.

### GC-MS Analyses of Derivatized Dicarboxylic Acids

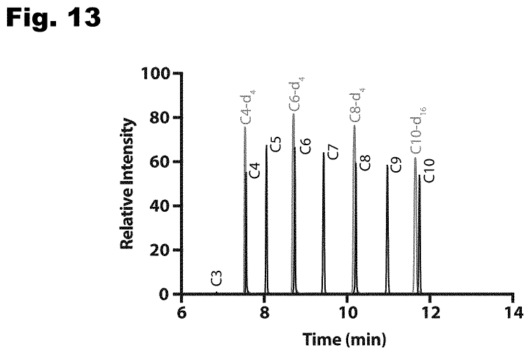

DCAs have two reactive carboxylic acid groups, making the parent mass M+2PFB. [M+1PFB]⁻carboxylate ions (m/z) were detected by injecting 1 μL derivatized extracts onto a 7890A GC system coupled to a 7000 MS Triple Quad (Agilent Technologies). Gas chromatography was performed over 21.2 min using a Phenomenex Zebron ZB-1MS capillary GC column (2×15 m length, 0.25 mm I.D., 0.50 μm film thickness) heated to 150° C. for 1.2 min, ramped to 270° C. at 20° C./min, and held for 2 min, then ramped to 340° C. at 10° C./min and held for 5 min. The temperature of the ion source was 200° C. and the temperature of the quadrupoles was 150° C. Single ion monitoring (SIM) was used to measure the [M+1PFB]⁻carboxylate ions after negative ion chemical ionization using methane gas. The coefficient of variation for detection of DCAs in urine samples is shown on Table 51. The reproducibility measures (SD) when repeating the entire preparation and GCMS of the same original sample was <20%; the SD when running the same sample by GCMS on consecutive days was <6%. The list of carboxylate ions (m/z) for non-deuterated and deuterated dicarboxylic acid standards, retention times, linear ranges, and limits of detection are shown in Table 2. The total ion chromatogram obtained from the GC/MS is shown in FIG. 13.

### Data and Statistical Analyses

Agilent MassHunter Workstation Software was used to analyze GC/MS data. A calibration curve was acquired prior to sample analysis and quality control standards were analyzed after each 10 samples. All samples were analyzed in triplicates. Peak integration was automatic for most fatty acids and manual integration was used in selected cases when automatic integration failed. The mass of DCA was examined normalized to volume, and then the percent distribution and proportion of the DCAs were determined. Utilizing the percentage reduced the coefficient of variation and also accounted for hydration as the percentages represent how each species relates to each other. Mann Whitney U tests were performed to determine significant differences in DCA levels between CH-NAT, CH-PAT, combined CH, and AD study participants. All data analyses were performed using GraphPad Prism software and data were considered statistically significant when P<0.05. Additionally, Spearman's rank correlation coefficients between DCA species and CSF levels of Ab and tau proteins were examined. Selected brain volumes were determined by MRI.

### Ratio Analysis

The hypothesis on which the example is powered is that the DCA lipid ratio in urine membranes at baseline will be smaller in participants who cognitively decline over 4 years compared to those who do not. The DCA lipids will be expressed as the ratio of urine C4-05 to C7-C9. Based on preliminary data, it estimated that the mean (SD) ratio to be 1.54 (1.22) in decliners and 1.99 (1.27) in non-decliners.

### Doctrine of Equivalents

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

TABLE 1
Demographic, clinical, and CSF/urine Biomarkers

| | | Clinical Classification | | | |
|---|---|---|---|---|---|
| | | | | CH | CH |
| CSF Aβ₄₂/Tau | | AD | AII CH | NAT | PAT |
| Classificatin | | (n = 24) | (n = 76a) | (n - 45a) | (n = 31) |
| Age + SD | | 79.2 ± 7.31 | 78.0 ± 6.45 | 77.3 ± 6.79 | 79.1 ± 5.88 |
| (age range) | | (62-91) | (63-91) | (63-90) | (68-91) |
| % Female | | 58.3% | 65.8% | 66.7% | 64.5% |
| ApoE Genoltype (b) | | | | | |
| 2/2 | | 0 | 0 | 0 | 0 |
| 2/3 | | 0 | 13 | 7 | 6 |
| 2/4 | | 0 | 2 | 0 | 2 |
| 3/3 | | 8 | 41 | 28 | 13 |
| 3/4 | | 4 | 15 | 6 | 9 |
| 4/4 | | 0 | 0 | 0 | 0 |
| BMI | | 25.45 ± 4.92 | 26.63 ± 5.03 | 26.81 ± 5.55 | 26.38 ± 4.24 |
| Education in Years | | 14.75 ± 2.71 | 16.55 ± 2.53** | 16.51 ± 2.39** | 16.61 ± 2.75* |
| CSF | Aβ₄₂ ± SD | 536.9 ± 236.5 | 759.1 ± 306.5** | 915.4 ± 247.6*** | 532.1 ± 234.6 |
| | (95% CI) [pg/mL] | (437.0-636.8) | (689.0-829.1) | (841.0-989.8) | (446.0-618.1) |
| | Total Tau ± SD | 417.1 ± 169.9 | 261.2 ± 148.5*** | 187.1 ± 71.05*** | 368.9 ± 165.8 |
| | (range) [pg/mL] | (345.3-488.8) | (227.3-295.2) | (165.7-208.4) | (308.0-429.7) |
| Urine | Total Protein ± SD | 182.7 ± 95.8 | 136.9 ± 72.62* | 135.6 ± 78.27* | 138.9 ± 64.75 |
| | (95% CI) [□g/mL] | (142.3-223.2) | (120.3-153.5) | (112.1-159.1) | (115.1-162.6) |
| | Creatinine ± SD | 1218.0 ± 720.4 | 1025.9 ± 550.5 | 1019.4 ± 558.3 | 1035.4 ± 548.0 |
| | (95% CI) [□g/mL] | (913.4-1522) | (900.1-1152) | (851.6-1187) | (834.5-1236) |
| | Albumin ± SD | 37.80 ± 25.44 | 25.97 ± 31.86** | 24.25 ± 23.21** | 28.42 ± 41.49* |
| | (95% CI) [□g/mL] | (27.06-48.55) | (18.64-33.30) | (17.19-31.30) | (13.20-43.64) |
| | UACR ± SD | 34.75 ± 23.21 | 28.66 ± 38.51* | 29.34 ± 45.06** | 27.69 ± 27.30 |
| | (95% CI) [mg/g] | (24.95-44.55) | (19.80-37.52) | (15.64-43.04) | (17.68-37.71) |

TABLE 2
Analytical parameters utilized to detect and quantify DCAs

| | | | | | Linear Range (ng) | | |
|---|---|---|---|---|---|---|---|
| Name | Carbon # | m/z | RT (min) | ISTD | LOD | Top | R² |
| Malonic acid | C3 | 283.0 | 6.85 | Succinic acid-d₄ | 0.587 | 3000 | 0.988 |
| Succinic acid-d₄ | C4 | 297.0 | 7.54 | — | N/A | N/A | N/A |
| Succinic acid | C4 | 301.0 | 7.56 | Succinic acid-d₄ | 0.156 | 750 | 0.971 |
| Glutaric acid | C5 | 311.0 | 8.06 | Adipic acid-d₄ | 0.140 | 750 | 0.974 |
| Adipic acid-d₄ | C6 | 329.0 | 8.72 | — | N/A | N/A | N/A |
| Adipic acid | C6 | 325.0 | 8.75 | Adipic acid-d₄ | 0.143 | 750 | 0.978 |
| Pimelic acid | C7 | 339.0 | 9.45 | Suberic acid-d₄ | 0.131 | 750 | 0.933 |
| Suberic acid-d₄ | C8 | 357.0 | 10.2 | — | N/A | N/A | N/A |
| Suberic acid | C8 | 353.0 | 10.2 | Suberic acid-d₄ | 0.148 | 750 | 0.987 |
| Azelaic acid | C9 | 367.0 | 11.0 | Sebacic acid-d₁₆ | 0.145 | 750 | 0.995 |
| Sebacic acid-d₁₆ | C10 | 397.0 | 11.7 | — | N/A | N/A | N/A |
| Sebacic acid | C10 | 381.0 | 11.8 | Sebacic acid-d₁₆ | 0.147 | 750 | 0.994 |

Carbon number (C3-C10), negative ion (m/z), retention time (RT), deuterated internal standards, detection linear range, and correlation (R²).

TABLE 3
Distribution, proportion, and intergroup comparisons of DCA species
normalized for urine volume (ng/mL) between clinical groups.

| | | | Mean ± SD (95% CI) | | | |
|---|---|---|---|---|---|---|
| Species | Classification | n | [ng/mL] | CV | p values | |
| Malonic acid | CH | 76 | 177.4 ± 155.8 (141.8-213.0) | 0.878 | CH vs AD | 0.0603 |
| (C3) | CH-NAT | 45 | 170.4 ± 111.7 (136.9-204) | 0.656 | CH-NAT vs CH-PAT | 0.6975 |
| | CH-PAT | 31 | 187.4 ± 205.4 (112.1-262.8) | 1.096 | CH-NAT vs AD | 0.1362 |
| | AD | 24 | 213.8 ± 126.2 (160.5-267.1) | 0.590 | CH-PAT vs AD | 0.1092 |
| Succinic | CH | 76 | 2911 ± 2213 (2406-3417) | 0.760 | CH vs AD | 0.8695 |
| acid | CH-NAT | 45 | 3074 ± 2398 (2354-3795) | 0.780 | CH-NAT vs CH-PAT | 0.6140 |
| (C4) | CH-PAT | 31 | 2674 ± 1924 (1968-3380) | 0.720 | CH-NAT vs AD | 0.9950 |
| | AD | 24 | 2661 ± 1471 (2040-3283) | 0.553 | CH-PAT vs AD | 0.7174 |
| Glutaric acid | CH | 76 | 395.9 ± 285.6 (330.6-461.1) | 0.723 | CH vs AD | 0.1938 |
| (C5) | CH-NAT | 45 | 398.0 ± 261.3 (319.5-476.5) | 0.657 | CH-NAT vs CH-PAT | 0.5847 |
| | CH-PAT | 31 | 392.7 ± 322.1 (274.6-510.9) | 0.820 | CH-NAT vs AD | 0.1396 |
| | AD | 24 | 284.3 ± 130.3 (229.3-339.3) | 0.458 | CH-PAT vs AD | 0.4635 |
| Adipic acid | CH | 76 | 971.3 ± 1187 (700.1-1243) | 1.222 | CH vs AD | 0.2356 |
| (C6) | CH-NAT | 45 | 926.1 ± 1192 (568.0-1284) | 1.287 | CH-NAT vs CH-PAT | 0.9916 |
| | CH-PAT | 31 | 1037 ± 1196 (598.3-1476) | 1.153 | CH-NAT vs AD | 0.2836 |
| | AD | 24 | 1002 ± 689.3 (711.0-1293) | 0.688 | CH-PAT vs AD | 0.2992 |
| Pimelic acid | CH | 76 | 642.7 ± 437.6 (542.8-742.7) | 0.681 | CH vs AD | [symbol] |
| (C7) | CH-NAT | 45 | 599.4 ± 419.9 (473.2-725.5) | 0.701 | CH-NAT vs CH-PAT | 0.1484 |
| | CH-PAT | 31 | 705.7 ± 461.7 (536.3-875.0) | 0.654 | CH-NAT vs AD | [symbol] |
| | AD | 24 | 1032 ± 760.0 (711.5-1353) | 0.736 | CH-PAT vs AD | [symbol] |
| Suberic acid | CH | 76 | 834.1 ± 567.8 (704.4-963.9) | 0.681 | CH vs AD | [symbol] |
| (C8) | CH-NAT | 45 | 803.8 ± 599.1 (623.8-983.8) | 0.745 | CH-NAT vs CH-PAT | 0.4120 |
| | CH-PAT | 31 | 878.1 ± 525.6 (685.3-1071) | 0.599 | CH-NAT vs AD | [symbol] |
| | AD | 24 | 1249 ± 878.4 (878.4-1620) | 0.703 | CH-PAT vs AD | 0.0850 |
| Azelaic acid | CH | 76 | 638.6 ± 685.3 (482-795.2) | 1.073 | CH vs AD | [symbol] |
| (C9) | CH-NAT | 45 | 544.1 ± 549.5 (379.0-709.2) | 1.010 | CH-NAT vs CH-PAT | 0.0757 |
| | CH-PAT | 31 | 775.8 ± 835.6 (469.3-1082) | 1.077 | CH-NAT vs AD | [symbol] |
| | AD | 24 | 1256 ± 1290 (711.3-1801) | 1.027 | CH-PAT vs AD | [symbol] |
| Sebacic acid | CH | 76 | 107.5 ± 131.1 (77.5-137.4) | 1.220 | CH vs AD | [symbol] |
| (C10) | CH-NAT | 45 | 108.8 ± 156.6 (61.76-155.8) | 1.439 | CH-NAT vs CH-PAT | 0.2381 |
| | CH-PAT | 31 | 105.5 ± 83.73 (74.79-136.2) | 0.794 | CH-NAT vs AD | [symbol] |
| | AD | 24 | 155.9 ± 161.8 (87.61-224.3) | 1.038 | CH-PAT vs AD | 0.1431 |
| Sum C3 − | CH | 76 | 6679 ± 3920 (5783-7574) | 0.587 | CH vs AD | 0.2230 |
| C10 | CH-NAT | 45 | 6625 ± 3982 (5429-7821) | 0.601 | CH-NAT vs CH-PAT | 0.8336 |
| | CH-PAT | 31 | 6756 ± 3894 (5328-8185) | 0.576 | CH-NAT vs AD | 0.2258 |
| | AD | 24 | 7855 ± 4539 (5939-9772) | 0.578 | CH-PAT vs AD | 0.3579 |

P values < 0.05 are shown in bold italics.

TABLE 4
Percent distribution, proportion, and intergroup comparison of DCA species
between clinical and biochemical groups.

| Species | Classification | n | % Mean ± SD (95% CI) | CV | p values | |
|---|---|---|---|---|---|---|
| Malonic acid | CH | 76 | 2.945 ± 1.701 (2.556-3.334) | 0.578 | CH vs AD | 0.6389 |
| (C3) | CH-NAT | 45 | 2.988 ± 1.586 (2.511-3.464) | 0.531 | CH-NAT vs CH-PAT | 0.4944 |
| | CH-PAT | 31 | 2.833 ± 1.881 (2.193-3.573) | 0.652 | CH-NAT vs AD | 0.9551 |
| | AD | 24 | 2.904 ± 1.268 (2.368-3.439) | 0.437 | CH-PAT vs AD | 0.3669 |
| Succinic | CH | 76 | 41.86 ± 12.13 (39.09-44.64) | 0.290 | CH vs AD | [symbol] |
| acid | CH-NAT | 45 | 44.12 ± 11.85 (40.56-47.68) | 0.269 | CH-NAT vs CH-PAT | 0.0869 |
| (C4) | CH-PAT | 31 | 38.59 ± 11.96 (34.21-42.98) | 0.310 | CH-NAT vs AD | [symbol] |
| | AD | 24 | 34.72 ± 10.59 (30.24-39.19) | 0.305 | CH-PAT vs AD | 0.1959 |
| Glutaric acid | CH | 76 | 6.346 ± 3.387 (5.572-7.120) | 0.534 | CH vs AD | [symbol] |
| (C5) | CH-NAT | 45 | 6.582 ± 3.528 (5.522-7.641) | 0.536 | CH-NAT vs CH-PAT | 0.4490 |
| | CH-PAT | 31 | 6.004 ± 3.198 (4.831-7.178) | 0.533 | CH-NAT vs AD | [symbol] |
| | AD | 24 | 4.353 ± 2.251 (3.420-5.303) | 0.517 | CH-PAT vs AD | 0.0653 |
| Adipic acid | CH | 76 | 13.76 ± 9.397 (11.61-15.91) | 0.683 | CH vs AD | 0.4636 |
| (C6) | CH-NAT | 45 | 13.76 ± 9.584 (10.88-16.63) | 0.697 | CH-NAT vs CH-PAT | 0.9916 |
| | CH-PAT | 31 | 13.76 ± 9.276 (10.36-17.16) | 0.674 | CH-NAT vs AD | 0.5049 |
| | AD | 24 | 13.10 ± 4.718 (11.11-15.09) | 0.360 | CH-PAT vs AD | 0.5275 |
| Pimelic acid | CH | 76 | 10.26 ± 3.793 (9.398-11.13) | 0.370 | CH vs AD | [symbol] |
| (C7) | CH-NAT | 45 | 9.749 ± 3.917 (8.573-10.93) | 0.402 | CH-NAT vs CH-PAT | 0.2609 |
| | CH-PAT | 31 | 11.01 ± 3.533 (9.716-12.31) | 0.321 | CH-NAT vs AD | [symbol] |
| | AD | 24 | 12.72 ± 2.912 (11.49-13.95) | 0.229 | CH-PAT vs AD | [symbol] |
| Suberic acid | CH | 76 | 13.25 ± 5.284 (12.04-14.46) | 0.400 | CH vs AD | [symbol] |
| (C8) | CH-NAT | 45 | 12.60 ± 4.788 (11.16-14.04) | 0.380 | CH-NAT vs CH-PAT | 0.3329 |
| | CH-PAT | 31 | 14.20 ± 5.885 (12.04-16.35) | 0.415 | CH-NAT vs AD | [symbol] |
| | AD | 24 | 15.61 ± 3.642 (14.08-17.17) | 0.233 | CH-PAT vs AD | 0.1339 |
| Azelaic acid | CH | 76 | 9.784 ± 6.603 (8.275-11.29) | 0.675 | CH vs AD | [symbol] |
| (C9) | CH-NAT | 45 | 8.475 ± 5.203 (6.912-10.04) | 0.614 | CH-NAT vs CH-PAT | 0.0689 |
| | CH-PAT | 31 | 11.68 ± 7.937 (8.772-14.59) | 0.679 | CH-NAT vs AD | [symbol] |
| | AD | 24 | 14.43 ± 7.770 (11.14-17.71) | 0.539 | CH-PAT vs AD | 0.1385 |
| Sebacic acid | CH | 76 | 1.788 ± 1.712 (1.396-2.179) | 0.958 | CH vs AD | 0.0721 |
| (C10) | CH-NAT | 45 | 1.734 ± 1.922 (1.157-2.311) | 1.109 | CH-NAT vs CH-PAT | 0.1806 |
| | CH-PAT | 31 | 1.865 ± 1.378 (1.360-2.370) | 0.739 | CH-NAT vs AD | [symbol] |
| | AD | 24 | 2.163 ± 1.763 (1.418-2.907) | 0.815 | CH-PAT vs AD | 0.5871 |
| Sum C4 + C5 | CH | 76 | 48.21 ± 13.05 (45.23-51.19) | 0.271 | CH vs AD | [symbol] |
| | CH-NAT | 45 | 50.70 ± 12.37 (46.98-54.42) | 0.244 | CH-NAT vs CH-PAT | 0.0722 |
| | CH-PAT | 31 | 44.60 ± 13.35 (39.70-49.50) | 0.299 | CH-NAT vs AD | [symbol] |
| | AD | 24 | 39.07 ± 11.14 (34.37-43.77) | 0.285 | CH-PAT vs AD | 0.1576 |
| Sum C7 − | CH | 76 | 35.09 ± 12.14 (32.31-37.86) | 0.346 | CH vs AD | [symbol] |
| C10 | CH-NAT | 45 | 32.56 ± 10.86 (29.30-35.82) | 0.334 | CH-NAT vs CH-PAT | [symbol] |
| | CH-PAT | 31 | 38.76 ± 13.12 (33.94-43.57) | 0.339 | CH-NAT vs AD | [symbol] |
| | AD | 24 | 44.92 ± 9.783 (40.79-49.06) | 0.218 | CH-PAT vs AD | 0.0604 |

What is claimed is:

1. A method to determine an individual's risk of Alzheimer's disease, comprising:
obtaining or having obtained a biological sample of an individual, wherein the biological sample contains dicarboxylic acids;
adding an internal standard of dicarboxylic acid molecules to the biological sample;
performing an assay on the biological sample to determine an amount of at least one long dicarboxylic acid species in the sample, wherein the determined amount of the at least one long dicarboxylic acid species indicates the individual's risk of Alzheimer's disease.

2. The method as in claim 1, wherein the biological sample is urine.

3. The method as in claim 1 or 2, wherein the assay is gas chromatography combined with mass spectrometry.

4. The method as in claim 3 further comprising: converting the dicarboxylic acids within the biological to dipentafluorobenzyl esters prior to performing gas chromatography combined with mass spectrometry.

5. The method any one of claims 1-4, wherein the internal standard of dicarboxylic acid molecules includes at least one of: succinic acid (C4), glutaric acid (C5), pimelic acid (C7), suberic (C8), azelaic acid (C9) and sebacic acid (C10).

6. The method as in any one of claims 1-5, wherein the internal standard of dicarboxylic acid molecules is a set of deuterated dicarboxylic acid molecules with known concentrations.

7. The method as in any one of claims 1-6, wherein the amount of at least one long dicarboxylic acid species is a relative amount to a set of one or more dicarboxylic acid species measured.

8. The method as in any one of claims 1-6, wherein the amount of at least one long dicarboxylic acid species is a concentration.

9. The method as in any one of claims 1-8, wherein the determined amount of the at least one long dicarboxylic acid species of the individual is greater than a threshold, and wherein the individual is determined to have a high risk of Alzheimer's disease based on the amount of the at least one long dicarboxylic acid species being greater than the threshold.

10. The method as in claim 9, wherein the threshold is based on the amount of the at least one long dicarboxylic acid species in a cognitively healthy population or in a population of individuals having Alzheimer's disease.

11. The method as in any one of claims 1-10, wherein the at least one long dicarboxylic acid species is pimelic acid (C7), suberic acid (C8), azelaic acid (C9), sebacic acid (C10), an unsaturated C7, C8, C9 or C10 dicarboxylic acid species, or a substituted C7, C8, C9 or C10 dicarboxylic acid species.

12. The method as in any one of claim 1-11 further comprising:
performing an assay on the biological sample to determine a relative amount of at least one short dicarboxylic acid species in the sample; and
determining a ratio of the relative amount of at least one long dicarboxylic acid species to the relative amount of at least one short dicarboxylic acid species, wherein the determined ratio indicates the individual's risk of Alzheimer's disease.

13. The method as in claim 12, wherein the determined ratio of the individual is greater than a threshold, and wherein the individual is determined to have a high risk of Alzheimer's disease based on the ratio being greater than the threshold.

14. The method as in claim 13, wherein the threshold is based on the ratio of the concentration of at least one long dicarboxylic acid species to the concentration of at least one short dicarboxylic acid species in a cognitively healthy population or in a population of individuals having Alzheimer's disease.

15. The method as in claim 12, 13, or 14, wherein the at least one short dicarboxylic acid specie is succinic acid (C4), glutaric acid (C5), an unsaturated C4 or C5 dicarboxylic acid specie, or a substituted C4 or C5 dicarboxylic acid specie.

16. The method as in any one of claims 1-15 further comprising:
obtaining or having obtained at least a second biological sample of the individual, wherein each of the obtained biological samples contain dicarboxylic acids and at least two biological samples were acquired two different time points;
adding an internal standard of dicarboxylic acid molecules to each biological sample; and
performing an assay on each of the biological samples to determine concentrations of at least one long dicarboxylic acid species, wherein the temporal change of the concentration of the at least one long dicarboxylic acid specie indicates the individual's risk of Alzheimer's disease.

17. The method as in claim 16, wherein an increase of the concentration of the long dicarboxylic acid species over time indicates a high risk of Alzheimer's disease.

18. The method as in claim 17, wherein the increase of the concentration of the long dicarboxylic acid species over time is greater than a threshold, indicating the high risk of Alzheimer's disease.

19. The method as in claim 18, wherein the threshold is based on the increase of the concentration of the long dicarboxylic acid species over time in a cognitively healthy population or in a population of individuals having Alzheimer's disease.

20. The method as in any one of claim 16-19 further comprising:
performing an assay on the biological samples to determine a concentration of at least one short dicarboxylic acid species in each sample; and
determining a ratio of the concentration of at least one long dicarboxylic acid species to the concentration of at least one short dicarboxylic acid species at each time point, wherein the temporal change of the determined ratios indicates the individual's risk of Alzheimer's disease.

21. The method as in claim 20, wherein an increase of the concentration of at least one long dicarboxylic acid species to the concentration of at least one short dicarboxylic acid species over time indicates a high risk of Alzheimer's disease.

22. The method as in claim 21, wherein the increase of the ratio of the concentration of at least one long dicarboxylic acid species to the concentration of at least one short dicarboxylic acid species over time is greater than a threshold, indicating the high risk of Alzheimer's disease.

23. The method as in claim 22, wherein the threshold is based on the increase of the ratio of the concentration of at least one long dicarboxylic acid species to the concentration of at least one short dicarboxylic acid species over time in a cognitively healthy population or in a population of individuals having Alzheimer's disease.

24. The method as in any one of claims 1-23 further comprising:
determining that the individual is at a high risk of Alzheimer's disease; and
administering a diagnostic test to further assess the individual for Alzheimer's disease.

25. The method as in claim 24, wherein the diagnostic test is a cognitive test, a neuropsychological test, or medical imaging.

26. The method as in claim 24, wherein the diagnostic test is the Mini Mental State Exam or the Montreal Cognitive Assessment.

27. The method as in any one of claims 1-26 further comprising:
determining that the individual is at a high risk of Alzheimer's disease; and
administering a cognitive exercise to the individual for Alzheimer's disease.

28. The method as in claim 27, wherein the cognitive exercise is an activity that utilizes at least one of memory, reasoning, or information processing.

29. The method as in any one of claims 1-28 further comprising:
determining that the individual is at a high risk of Alzheimer's disease; and
administering a medication to the individual for Alzheimer's disease.

30. The method as in claim 29, wherein the medication is a cholinesterase inhibitor or a N-methyl D-aspartate receptor agonist.
